(19) United States
(12) Patent Application Publication
(10) Pub. No.: US 2018/0157798 A1
(43) Pub. Date: Jun. 7, 2018

(54) BLOOD TRACKING AND DELIVERY DEVICES AND METHODS

(71) Applicant: MUSTAFA NOURALDEEN ALBOKHARY, PENSACOLA, FL (US)

(72) Inventor: MUSTAFA NOURALDEEN ALBOKHARY, PENSACOLA, FL (US)

(21) Appl. No.: 15/371,275

(22) Filed: Dec. 7, 2016

(51) Int. Cl. G06F 19/00; B64C 39/02; G01C 21/00

(52) U.S. Cl. CPC G06F 19/327; B64C 2201/128; G01C 21/00; B64C 39/024

(57) ABSTRACT

A blood tracking and delivery system server has processing circuitry configured to receive blood data at a blood database center, wherein a) the blood data is sorted and indexed into a plurality of database tables, b) each entry of a blood donor data table is cross-referenced to respective indexed entries of a blood classification data table, and c) each indexed entry of a blood recipient data table is cross-referenced to respective indexed entries the blood classification data table; receive a blood supply request; cross-match the blood criteria for the recipient with donor blood criteria from the blood donor data tables; and determine a delivery route for the blood supply cross-matched to the blood recipient destination, where an unmanned aerial vehicle UAV is configured to deliver the blood supply to the blood recipient destination.

### GRANT OF NON-EXCLUSIVE RIGHT

This application was prepared with financial support from the Saudi Arabian Cultural Mission, and in consideration therefore the present inventor(s) has granted The Kingdom of Saudi Arabia a non-exclusive right to practice the present invention in the United States.

### BACKGROUND

Many surgeries are canceled each year due to a lack of information about blood availability. Cancellations due to difficulties in finding appropriate blood types in adequate amounts for surgeries result in a decrease in hospital capacity and an inefficiency of medical personnel. In many instances, patients schedule surgeries months in advance. Doctors and supporting medical personnel can be occupied for several hours for each individual operation.

Natural disasters and other widespread calamities typically involve injuries to hundreds or thousands of individuals, which may need urgent blood transfusions. In many cases, the nearest hospital cannot cover all of the immediate requests for blood transfusions due to the lack of information regarding blood availability. In addition, the duration of requests for blood types from the nearest hospital can be a lengthy process, as well as arrival of blood from a particular blood facility to the disaster area.

In an urgent surgical situation or a natural disaster situation, it is necessary to have an adequate supply of particular blood types available and nearby. If a patient is taken to a hospital in an emergency situation and the hospital has no blood that matches the patient, the patient is either transferred to another facility or the blood is requested from another facility. This can consume a great deal of time and endanger the patient. Therefore, quick access to information about blood availability, as well as an efficient mobility of blood transfer from one facility to another is critical for a patient's well-being.

The “background” description provided herein is for the purpose of generally presenting the context of the disclosure. Work of the presently named inventors, to the extent it is described in this background section, as well as aspects of the description which may not otherwise qualify as conventional at the time of filing, are neither expressly nor impliedly admitted as conventional against the present disclosure.

### SUMMARY

One embodiment includes a blood tracking and delivery system, which includes a blood tracking and delivery system server having processing circuitry configured to receive blood data at a blood database center, wherein a) the blood data is sorted and indexed into a plurality of database tables, b) each entry of a blood donor data table is cross-referenced to respective indexed entries of at least one blood classification data table, and c) each indexed entry of a blood recipient data table is cross-referenced to respective indexed entries of the at least one blood classification data table; receive a blood supply request from at least one computing device, the blood supply request including blood criteria for a recipient; cross-match the blood criteria for the recipient associated with the blood supply request with donor blood criteria from one of the blood donor data tables; determine a location of a blood supply cross-matched to the blood supply request based on a distance between the blood supply recipient and the cross-matched blood supply; determine a delivery route for the blood supply cross-matched to the blood supply request to a blood recipient destination that corresponds to the determined location, wherein an unmanned aerial vehicle (UAV) is configured to deliver the blood supply to the blood recipient destination.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is an overview of an exemplary blood tracking and delivery system according to one embodiment;

FIG. 1B illustrates a computing device view of a blood tracking and delivery system according to one embodiment;

FIG. 1C illustrates a system view of blood tracking and delivery system according to one embodiment;

FIGS. 2A-2C are tables illustrating a system of blood groups for Systems 001 through 030 and their respective antigens for Antigen Numbers 001 through 012 according to one embodiment;

FIG. 3 is a table illustrating Systems 002, 004-006, 010, and 021 for Antigen Numbers 013 through 024, and Systems 002, 004, and 006 for Antigen Numbers 025 through 035 according to one embodiment;

FIG. 4 is a table illustrating Systems 002 and 004 for Antigen Numbers 036 through 046, and System 004 for Antigen Numbers 047 through 057 according to one embodiment;

FIG. 5 is a table illustrating a cross-matching of a particular patient or recipient with an acceptable donor according to one embodiment;

FIG. 6 is a block diagram illustrating the relationships of blood database tables according to one embodiment;

FIG. 7A is a block diagram illustrating an exemplary blood web portal according to one embodiment;

FIG. 7B illustrates multiple database servers serving a particular geographical region in which blood is available according to one embodiment;

FIG. 7C illustrates multiple database servers serving a particular type of blood center according to one embodiment;

FIG. 7D illustrates an exemplary progression for a blood tracking & delivery interface for a mobile application according to one embodiment;

FIG. 8 is a block diagram of an exemplary blood delivery logistical system according to one embodiment;

FIG. 9 is a block diagram of an exemplary computing system according to one embodiment;

FIG. 10 is a schematic diagram of an exemplary data processing system according to one embodiment;

FIG. 11 is a block diagram of an exemplary CPU according to one embodiment;

and

FIG. 12 is a flowchart for an exemplary method of tracking and delivering blood according to one embodiment.

### DETAILED DESCRIPTION

The following descriptions are meant to further clarify the present disclosure by giving specific examples and embodiments of the disclosure. These embodiments are meant to be illustrative rather than exhaustive. The full scope of the disclosure is not limited to any particular embodiment disclosed in this specification, but rather is defined by the claims.

In the interest of clarity, not all of the features of the implementations described herein are shown and described in detail. It will be appreciated that in the development of any such actual implementation, numerous implementation-specific decisions need to be made in order to achieve the developer's specific goals, such as compliance with application- and business-related constraints, and that these specific goals will vary from one implementation to another and from one developer to another.

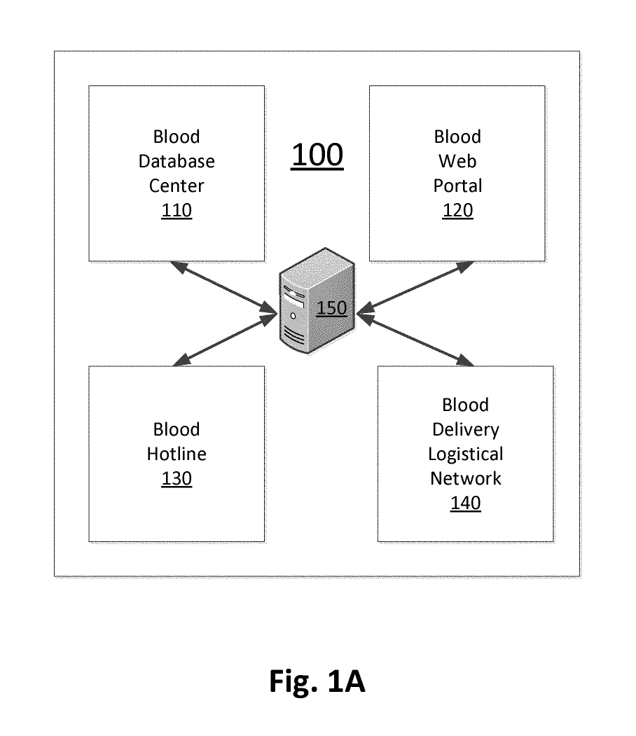

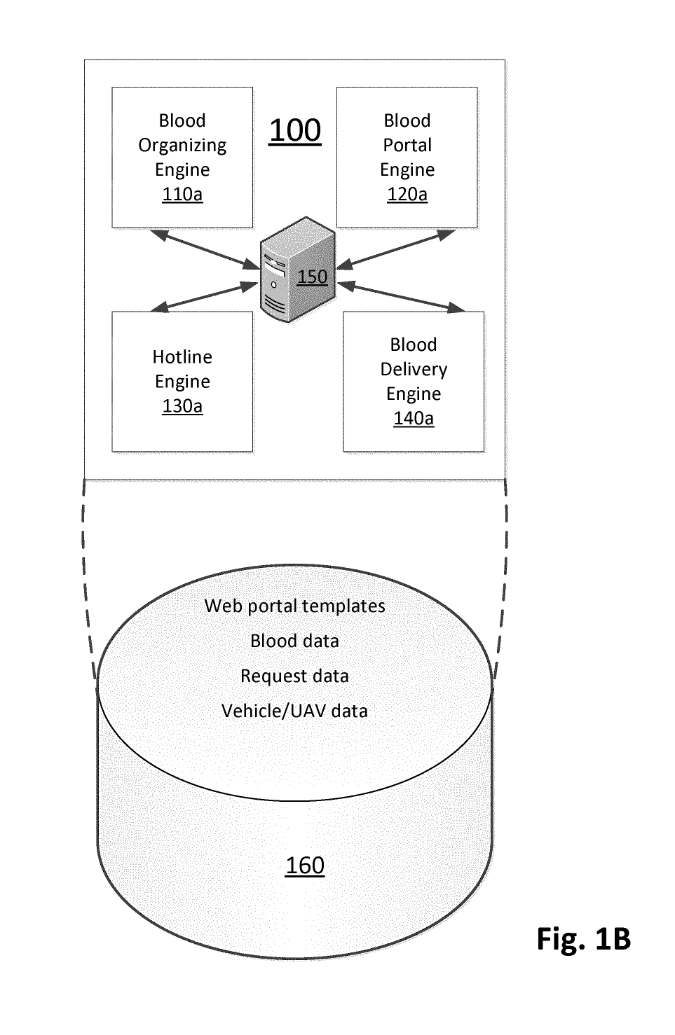

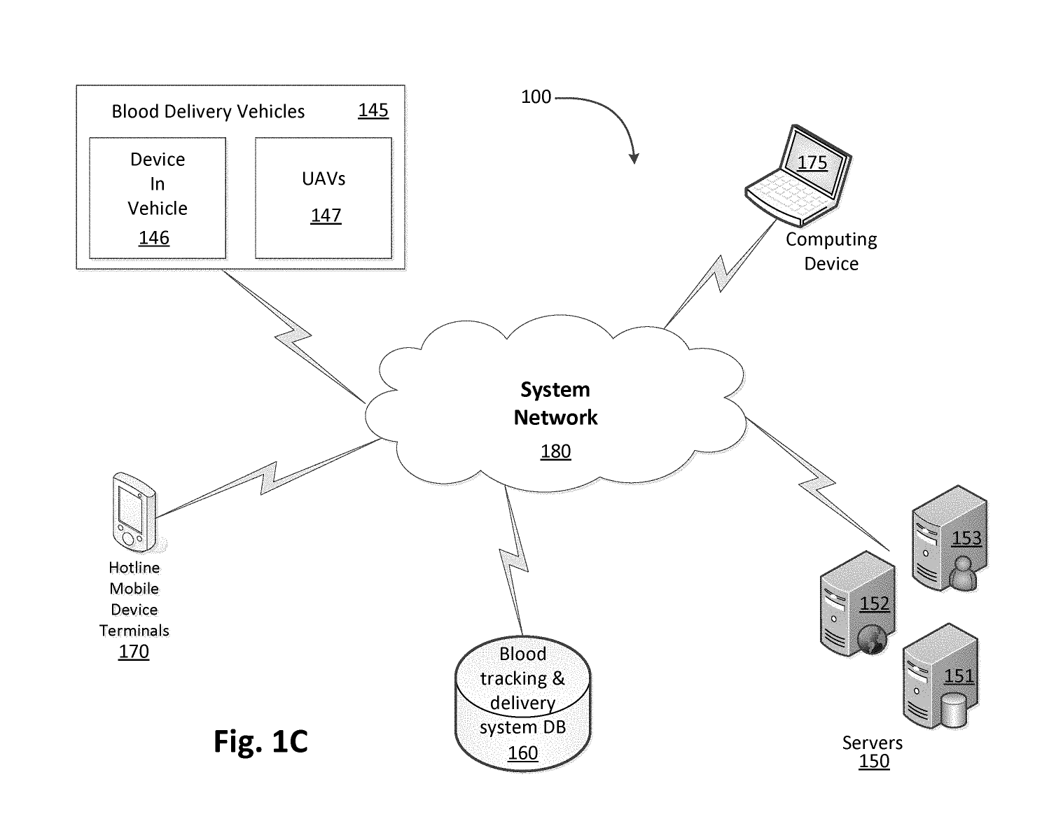

Embodiments herein describe systems and methods for tracking and delivering blood supplies efficiently and accurately. FIG. 1A is an overview of a blood tracking and delivery system 100, which includes a blood database center 110, a blood web portal 120, a blood hotline 130, and a blood delivery logistical network 140, all of which are interconnected by a system server 150. A more detailed description of the blood tracking and delivery system 100 is illustrated in FIG. 1C.

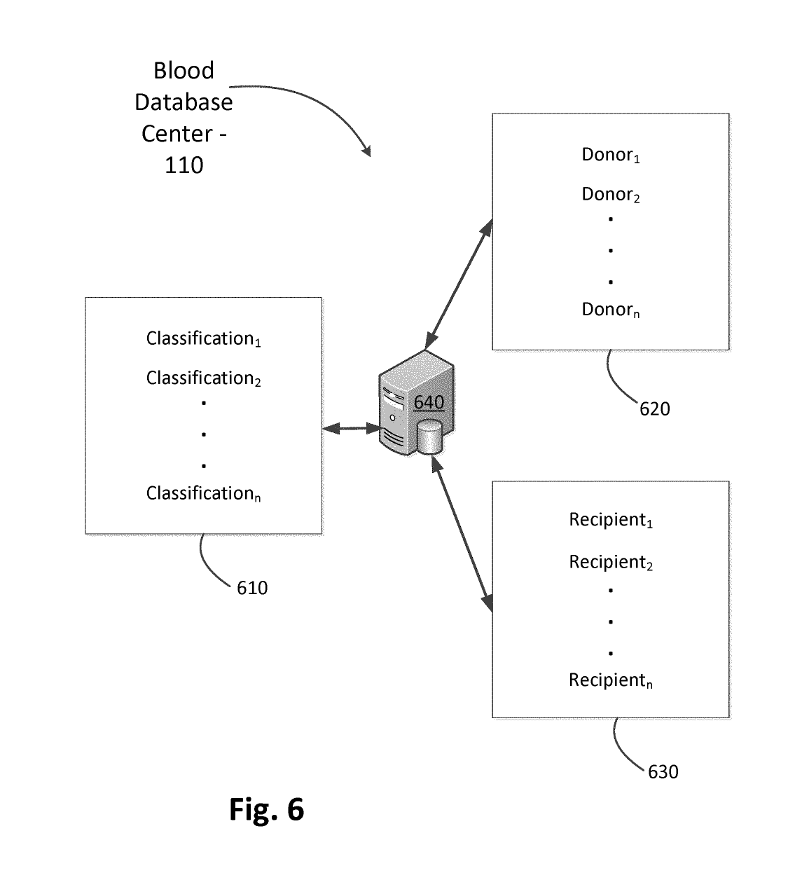

The blood database center 110 includes up-to-date information about the type and quantity of blood located in different facilities, such as hospitals and blood banks. Information includes, but is not limited to blood type and the presence of antibodies and/or antigens. A detailed description of blood database center 110 is given herein with reference to FIG. 6.

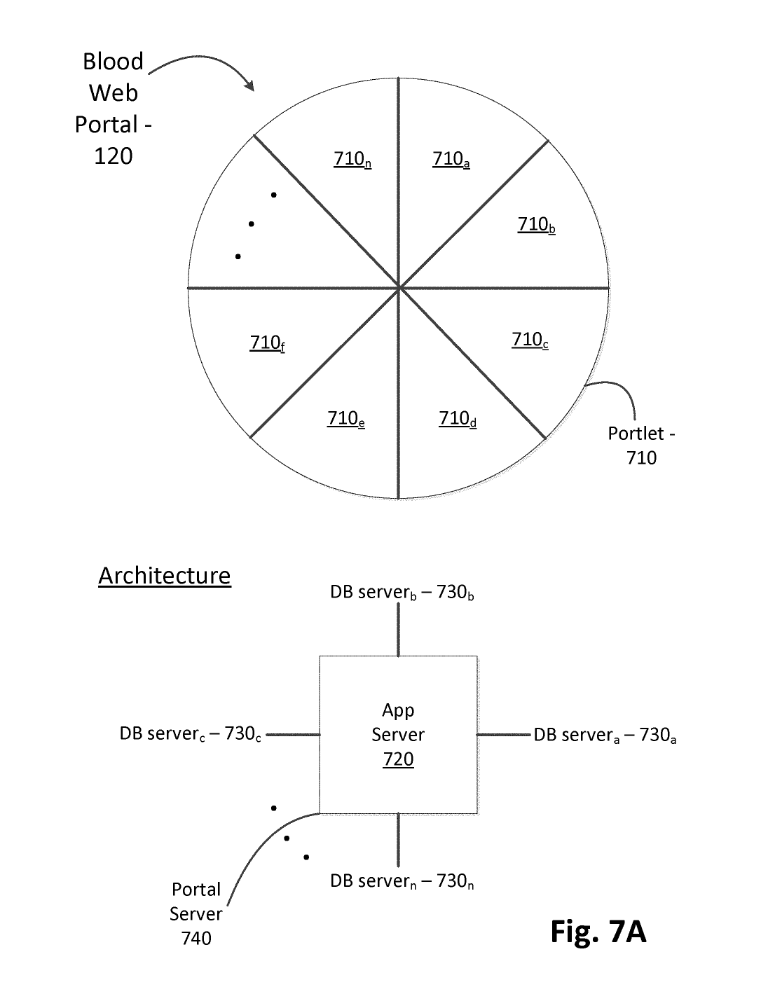

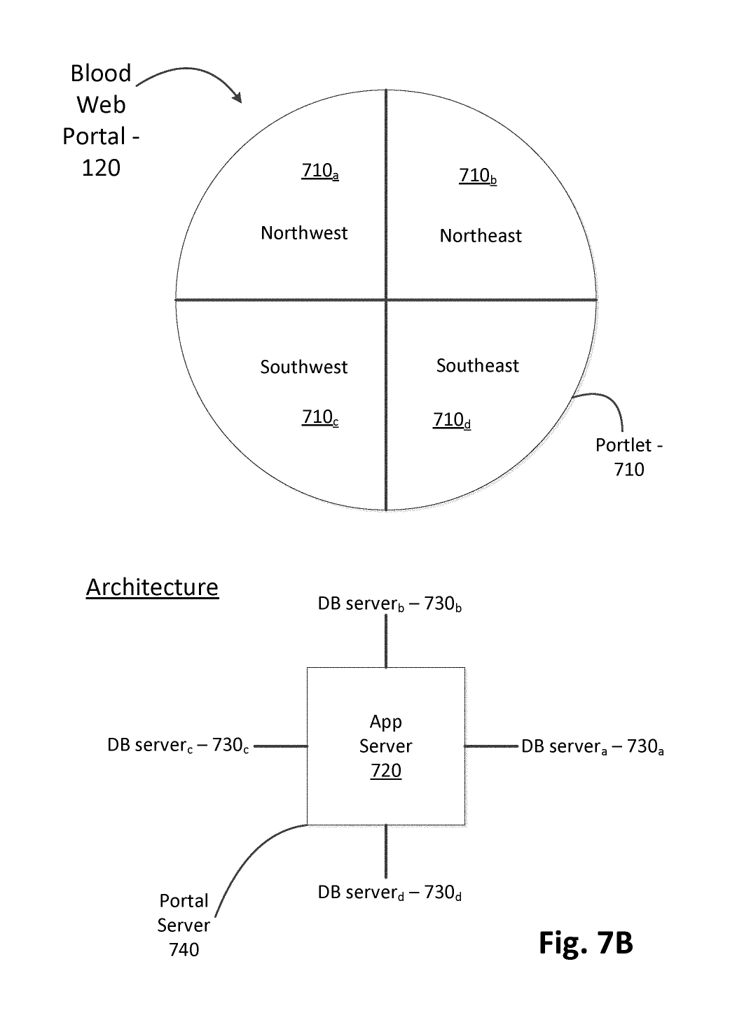

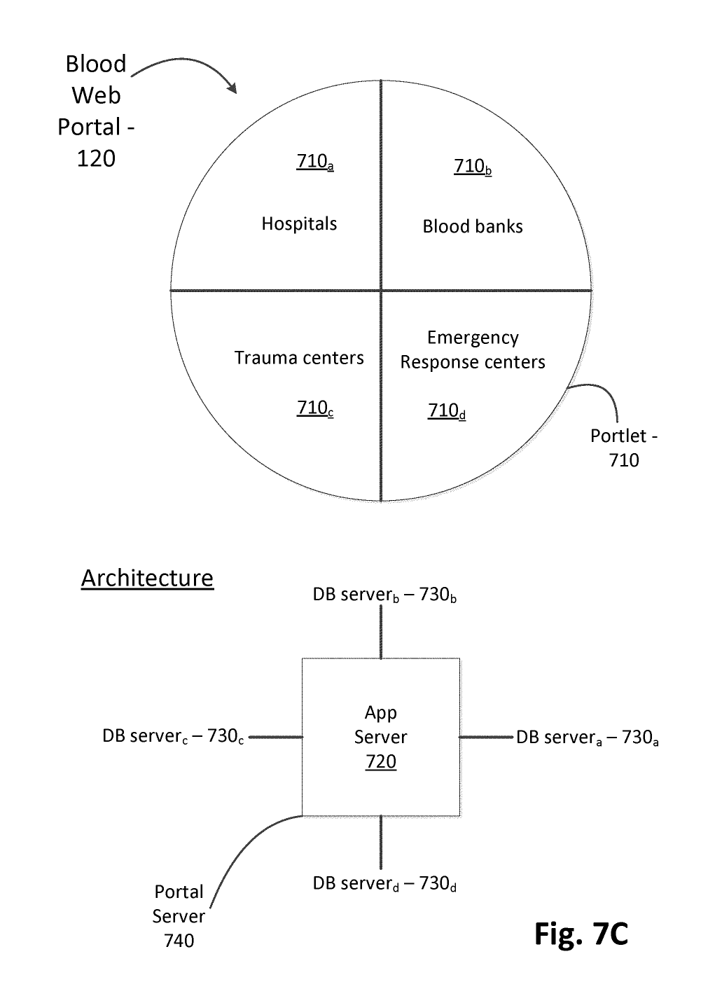

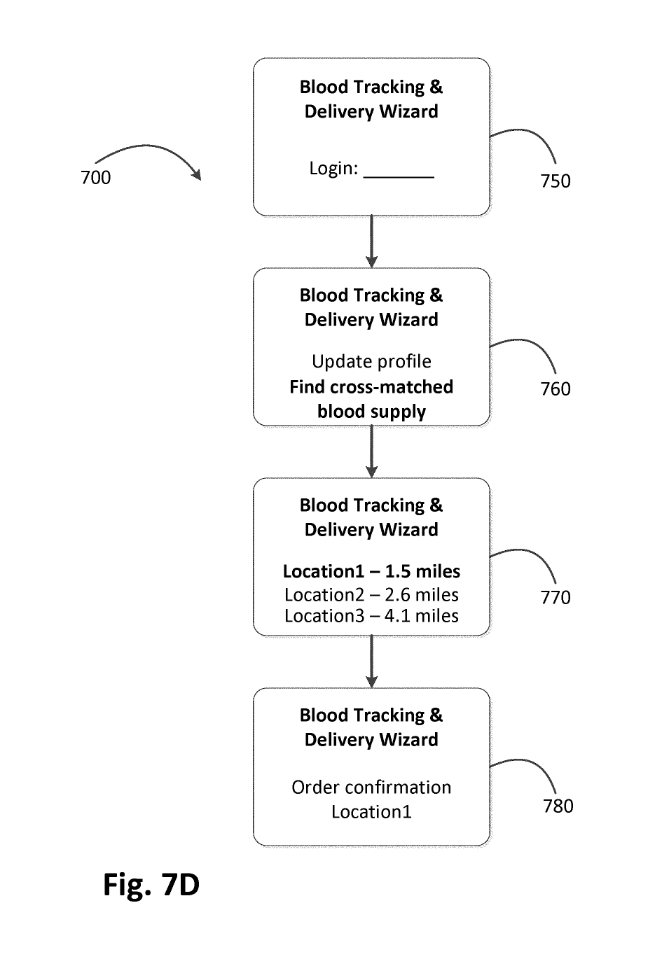

The blood web portal 120 is an interface between different components of the blood tracking and delivery system 100, which provides geographical information about where specific blood supplies are available that will satisfy the medical needs of a blood supply request. A mobile version is also available to show the availability of blood in different locations and the nearest hospital in which the requested blood is available. A detailed description of blood web portal 120 is given herein with reference to FIG. 7A.

The blood hotline 130 provides instant access for locating the nearest hospital or facility with the desired blood. This can be used in addition to or in lieu of the web portal when Internet access or a necessary application is not available.

The blood hotline 130 of the blood tracking and delivery system 100 provides direct access of blood availability via a communication link that can be an alternative to the blood web portal 120. The blood hotline 130 can include multiple communication links, such as an Internet link, a SMS texting link, a telephone link for voice or fax communication, and a social media link. The blood hotline 130 can be used in the event of slow or unavailable Internet access.

In one embodiment, the blood hotline 130 can provide continuous access as a notification system for both donors and recipients. In a first example, a cross-matched donor and recipient are notified of the match and the associated details, such as a location of the identified cross-matched blood to the recipient. In a second example, a blood hotline notification is also made to the recipient when a match has not been located within a predetermined period of time.

The blood hotline 130 can also include automated interactive two-way communication capabilities. For example, direct access can be provided for an individual with a registered blood account in the blood database center 110 to be immediately cross-matched with an available blood supply of a donor, via the system server 150. For a non-registered individual, a series of questions can be requested from the user, where the associated response can include a determination of subsequent questions that are asked of the user interacting with the blood hotline 130. In one embodiment, the ABO blood type and the Rh blood type are asked of the user first. For any unknown or indeterminate responses, transfer to a live assistant can be provided. A live assistant can also be reached in response to a user-initiated action. The blood hotline 130 can also retrieve geospatial position (GPS) information from a device being used by a user to match the user's position to a closest blood center meeting the user's requirements.

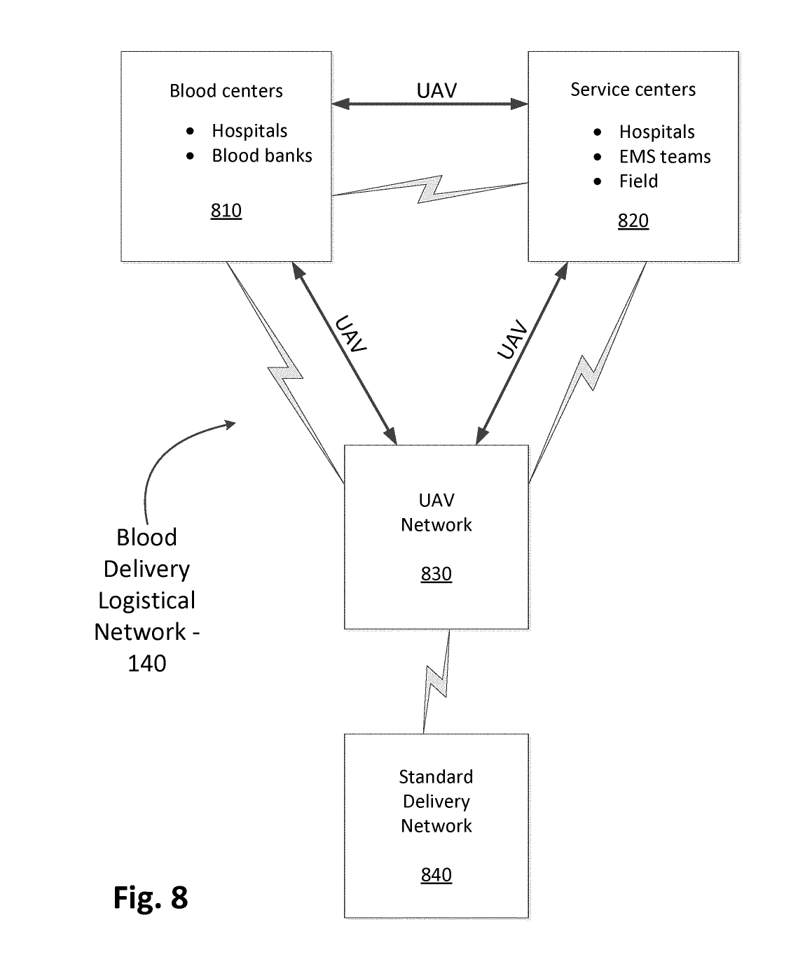

The blood delivery logistical network 140 provides a network of unmanned aerial vehicles (UAVs), also known as drones, for delivery of a blood supply from a source to a destination. The blood delivery logistical system is particularly valuable in a large urban area where the population is dense, and in disaster-hit regions in which conventional transportation routes may be temporarily unavailable. The blood delivery logistical network 140 is described in more detail herein with reference to FIG. 8.

FIG. 1B illustrates a computing device view of blood tracking and delivery system 100. The blood tracking and delivery system 100 includes one or more engines or modules that perform processes associated with receiving collected blood data, organizing the blood data based on type, location, etc., receiving requests for blood supplies, processing the requests for the blood supplies, and dispatching one or more blood delivery vehicles to deliver the blood supplies. References to the engines or modules throughout the disclosure are meant to refer to software processes executed by circuitry of one or more processing circuits, which can also be referred to interchangeably as processing circuitry. A blood organizing engine 110a sorts and organizes blood data received at the blood database center 110. Blood data includes, but is not limited to blood classification data, blood donor data, blood recipient data, and blood location data. Blood data can be received from users, donors, recipients, and blood suppliers via system server 150.

A blood portal engine 120a organizes the information sources that are represented by the blood web portal 120. Information sources include, but are not limited to hospitals, blood banks, trauma centers, and emergency response centers. Blood portal engine 120a formats each information source into a uniform format for display via one or more user interface screens and controls access to other components of the blood tracking and delivery system 100, via system server 150.

A hotline engine 130a receives, organizes, and transmits communications directly to a designated destination, via system server 150. A designated destination can include hotline transmissions to the blood database center 110 for a blood type inquiry, the blood web portal 120 for a blood location inquiry, or the blood delivery logistical network 140 for a delivery inquiry.

A blood delivery engine 140a organizes the logistical transportation for delivery of blood supplies for the blood delivery logistical network 140. A blood delivery request can be received at the blood delivery engine 140a from the blood portal engine 120a or the hotline engine 130a, via system server 150.

Each of the blood organizing engine 110a, the blood portal engine 120a, the hotline engine 130a, and the blood delivery engine 140a transmit data to and from a blood tracking and delivery system database 160. Blood tracking and delivery system database 160 includes, but is not limited to web portal templates from the blood web portal 120, blood data from the blood database center 110, request data from the blood web portal 120 and/or the blood hotline 130, and vehicle and/or UAV data from the blood delivery logistical network 140. Data transfer is implemented via system server 150.

FIG. 1C illustrates a system view of blood tracking and delivery system 100. Servers 150 include, but are not limited to a database server 151, a web server 152, and a real-time server 153. The database server 151 can be used in conjunction with the blood database center 110, the web server 152 can be used in conjunction with the blood web portal 120, and the real-time server 153 can be used with either of the blood hotline 130 or the blood delivery logistical network 140.

The blood tracking and delivery system database 160 includes, but is not limited to web portal templates from the blood web portal 120, blood data from the blood database center 110, request data from the blood web portal 120 and/or the blood hotline 130, and vehicle and/or UAV data from the blood delivery logistical network 140.

Blood delivery vehicles 145, such as vehicles with a communications device 146 and UAVs 147 execute the logistical transportation paths determined by the blood delivery logistical network 140. One or more computing devices 175 and one or more hotline mobile device terminals 170 execute communications between different components of the blood tracking and delivery system 100, via a system network 180. System network 180 includes, but is not limited to wired and wireless networks, cloud networks, the Internet, intranets, extranets, wide-area networks (WANs), and local-area networks (LANs).

Blood tracking and delivery system 100 includes various devices with processing circuitry for executing functions of each of the blood database center 110, the blood web portal 120, the blood hotline 130, and the blood delivery logistical network 140, as well as integration of each component with one or more other components of the blood tracking and delivery system 100. In one embodiment, special purpose processing circuitry is configured to receive a blood supply request from a computing device associated with a user, cross-match blood criteria of a recipient of the blood supply request with blood criteria of a donor via the blood database center 110, determine a location of the cross-matched blood supply via the blood availability network of the blood web portal 120, and determine a delivery route of the cross-matched blood supply from the blood center to the recipient at the service center via the blood delivery logistical network 140. However, special purpose processing circuitry also exists throughout the blood delivery logistical network 140 and is responsible for execution of numerous functions therein.

There are currently about thirty-five different blood groups and over six hundred blood group antigens. The most common blood group is the ABO group, which includes A-type blood, B-type blood, AB-type blood, and O-type blood. Subgroups of blood types also exist.

An antibody is a large Y-shaped protein produced primarily by plasma cells that is used by the immune system to identify and neutralize pathogens, such as bacteria and viruses. The antibody recognizes a unique antigen molecule of a harmful agent. Each tip of the Y-shaped antibody contains a paratope that is specific for one particular epitope on an antigen, allowing the two structures to bind together. This binding mechanism allows the antibody to tag a microbe or an infected cell for attack by other parts of the immune system to neutralize its target directly.

The second most common blood group is the Rh blood group. There are approximately fifty defined Rh blood group antigens. The most common antigens are D, C, c, E, and e, with the D antigen being the most prevalent. When there is no antigen specified for an Rh blood group, it is presumed to be the D antigen. An Rh blood group antigen is either present (Rh-positive) or not present (Rh-negative).

For a vast share of the population, the ABO type and the Rh factor are the only blood group concerns for donating or receiving blood. However, there can be several other critical considerations for many individuals.

Blood cross-matching is a process used to determine a compatible match between a donor and a recipient. Blood cross-matching can be performed between donor database tables and recipient database tables via a database server, such as database server 640 illustrated in FIG. 6. An initial cross-matching consideration includes ABO blood typing and Rh blood typing of the donor and the recipient, as well as an antibody screen of the recipient. A negative antibody screen usually means that there are no active red blood cell atypical antibodies present in the donor, or they are below a detectable level of current testing methods. In a major cross-match, recipient serum is tested against donor cells to determine if the recipient has pre-formed antibodies against any antigens on the donor's cells. In a minor cross-match, recipient red cells are tested against donor serum to detect donor antibodies directed against a patient's antigens.

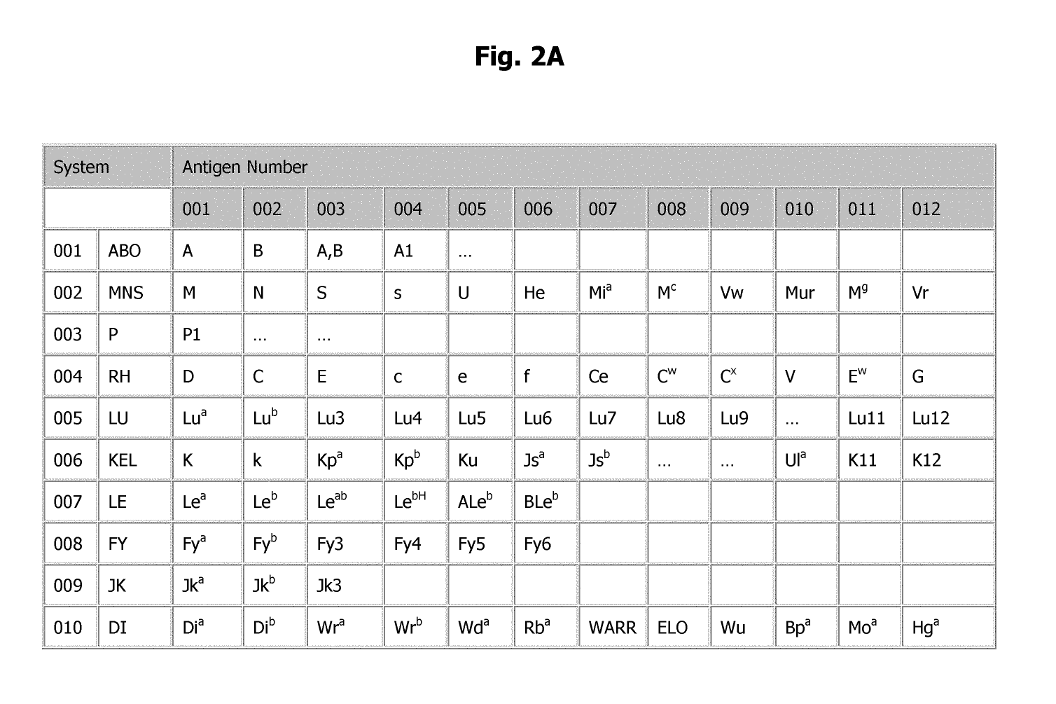

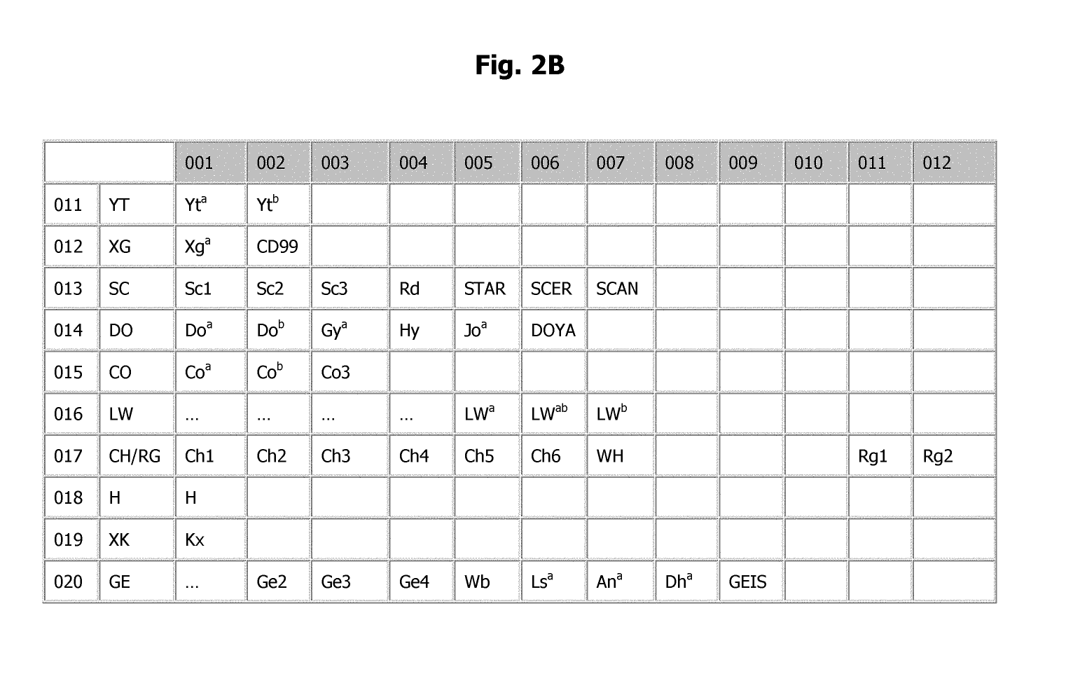

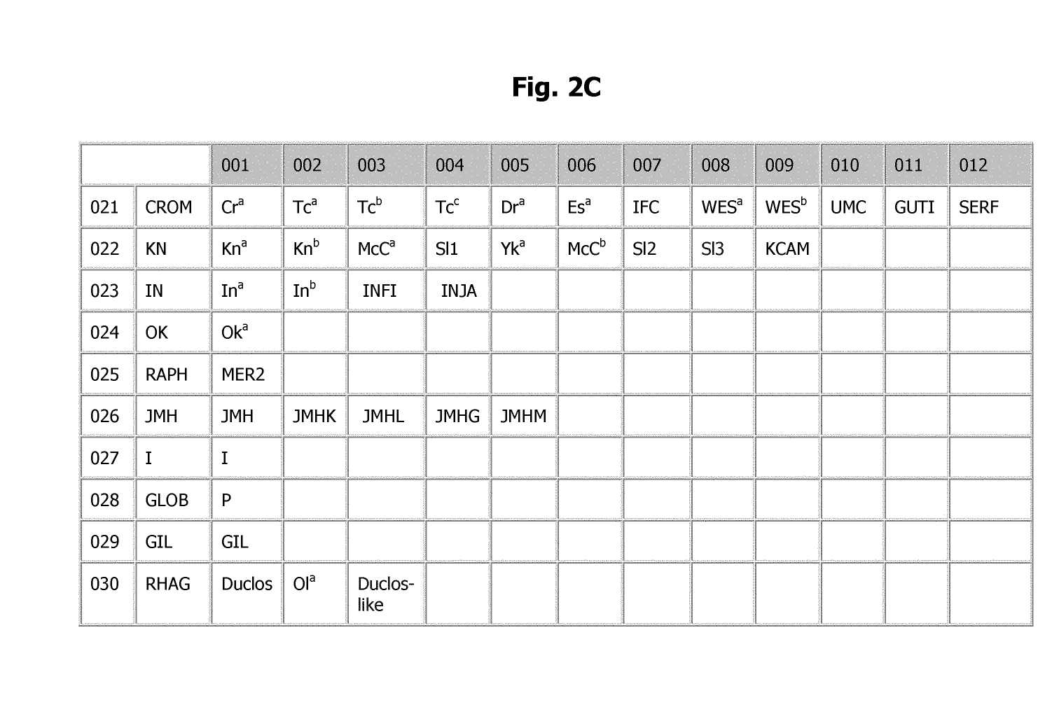

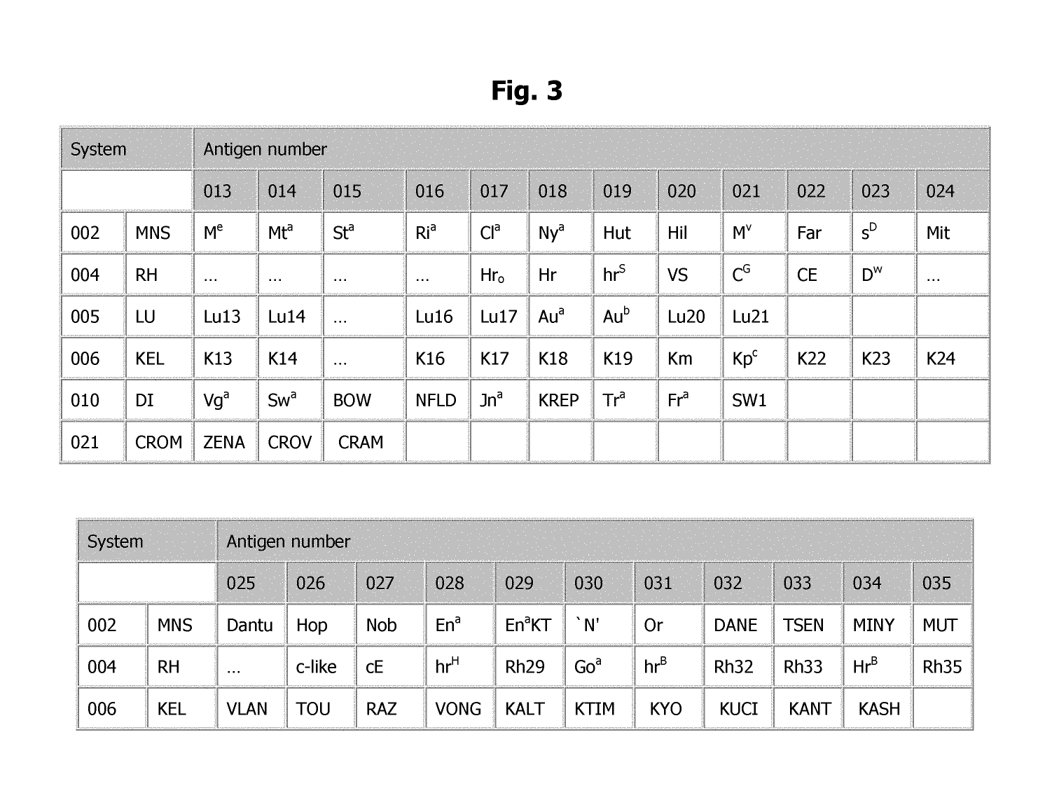

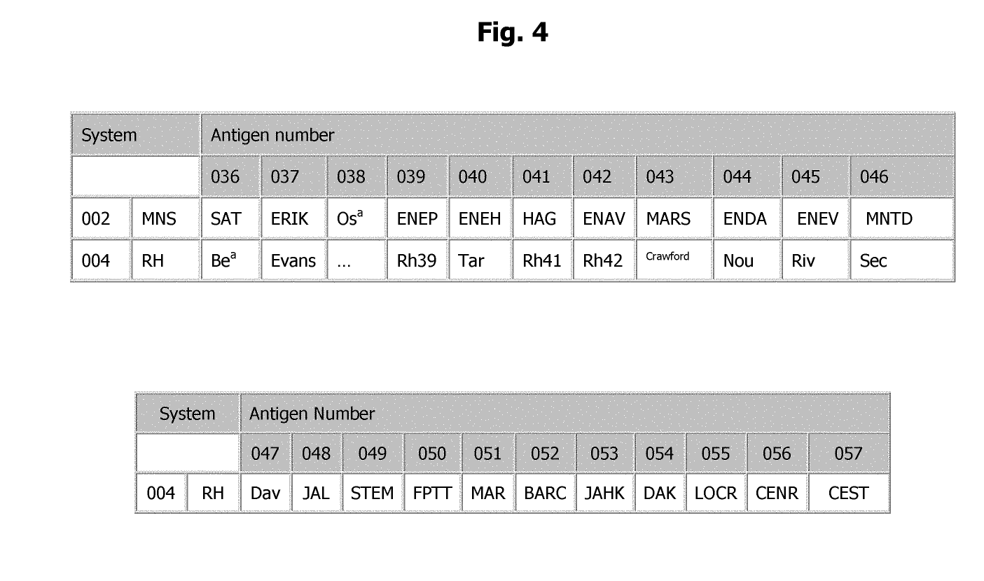

FIGS. 2A, 2B, and 2C are tables illustrating a system of blood groups for Systems 001 through 030 and their respective antigens for Antigen Numbers 001 through 012. FIG. 3 is a table illustrating Antigen Numbers 013 through 024 for Systems 002, 004-006, 010, and 021. FIG. 3 also illustrates Antigen Numbers 025 through 035 for Systems 002, 004, and 006. FIG. 4 is a table illustrating Antigen Numbers 036 through 046 for Systems 002 and 004. FIG. 4 also illustrates Antigen Numbers 047 through 057 for System 004.

In one embodiment, data from the above-cited tables can be included in one or more database classification tables. This data can be used to cross-match data for donors and recipients contained in one or more other databases, via database server 640. FIG. 6 is a block diagram of blood database center 110, which illustrates the relationships of blood database tables. Blood classification data tables 610 include the blood system and antigen data tables, as illustrated in FIGS. 2A-2C, 3, and 4. As discussed herein, database server 640 organizes and clusters the blood classification data tables 610 by various criteria, such as frequency of use, links to a disease or medical condition, and/or geographical location association.

FIG. 6 also illustrates one or more blood donor data tables 620, which include blood criteria of each blood donor, such as the blood system, antibodies, and antigens of each respective donor. The criteria of each blood donor in the blood donor data tables 620 are cross-referenced via database server 640 to all applicable blood system and antigen entries in the blood classification data tables 610.

FIG. 6 also illustrates one or more blood recipient data tables 630, which include blood criteria of each blood recipient, such as the blood system, antibodies, and antigens of each respective recipient. Blood recipient data tables 630 also include any requirements concerning the absence of any blood systems and/or antigens. Database server 640 cross-references the criteria of each blood recipient in the blood recipient data tables 630 to all applicable blood system and antigen entries in the blood classification data tables 610.

In another embodiment, the single database table listing all blood groups and their respective antigens can be divided into multiple database tables, such as a first database table for Systems 001-010, a second database table for Systems 011-020, and a third database table for Systems 021-030. The single database table or the set of three database tables representing all blood groups and their respective antigens are organized by database server 640.

In a second embodiment, the system of blood groups and their respective antigens can be sorted by database server 640 in descending order according to their expected usage of data retrieval. For example, the ABO system (System 001) and the Rh system (System 004) are the most commonly used systems in cross-matching donors and recipients. Therefore, they can be listed first, followed by the remaining blood systems in descending order according to their expected usage. If multiple database tables are used, the ABO system and the Rh system can be configured within a first database table, followed by one or more other database tables, according to their expected usage. In an alternative embodiment, the ABO system and the Rh system database table can be included within the cache system of a central processing system of the blood web portal 120 for faster retrieval.

In a third embodiment, blood group database tables can be clustered according to a particular disease or medical condition via database server 640. The clusters can be formed according to blood systems or antigens. For example, certain illnesses or conditions require the absence of one or more specific antigens. Hematological disorders include, but are not limited to sickle-cell anemia, thalassemia, leukemia, polycythemia vera, thrombocytosis, myelodysplastic syndrome, hemophilia, HIV, Hepatitis B and C, malaria, and trypanosomiasis. Blood recipients having a hematological disorder usually require more than the correct ABO and Rh System blood. Blood database center 110 can include several blood database tables clustered according to a medical disorder or condition for faster cross-matching of a donor and recipient.’

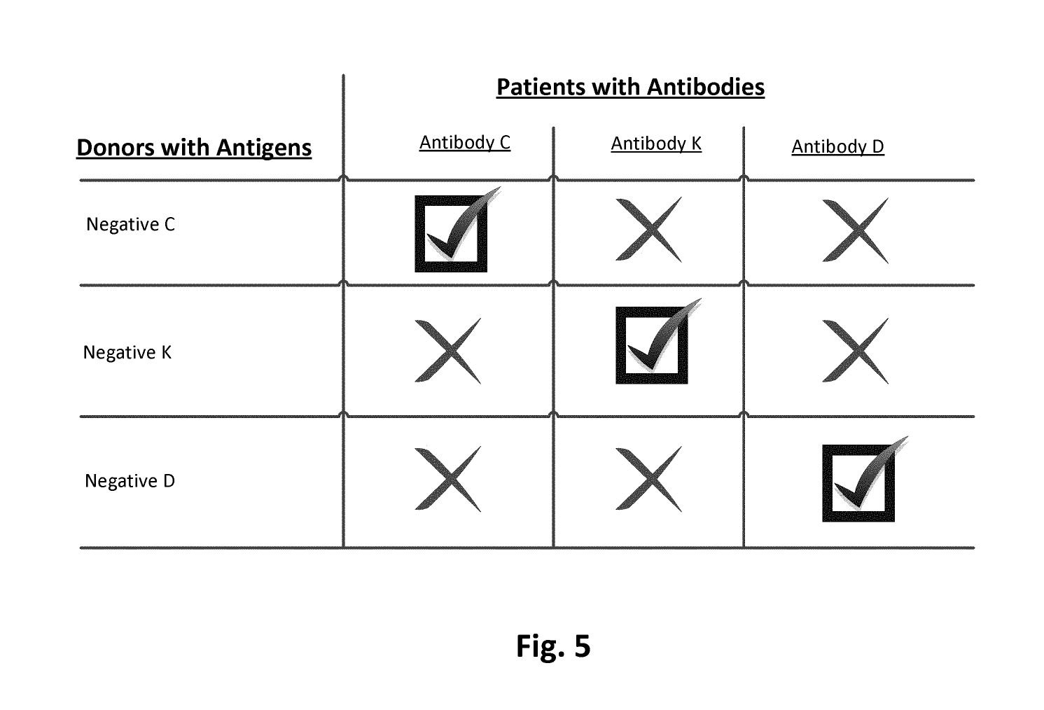

FIG. 5 is a table illustrating a cross-matching of a particular patient or recipient with an acceptable donor. In the first line of the table, a first patient having a C antibody could only accept blood from a donor that is negative for a C antigen. A second patient having a K antibody could only accept blood from a donor that is negative for a K antigen. A third patient having a D antibody could only accept blood from a donor that is negative for a D antigen.

In many cases, a patient may have more than one antibody. For example, a patient may have a C antibody and a D antibody. Therefore, a donor that is negative for both a C antigen and a D antigen would be needed to provide a blood supply to the patient.

FIG. 5 illustrates just three antibodies and three antigens for simplicity and ease of illustration. However, an actual table would likely have several donor antigens listed and several patient antibodies listed. In addition, the “Donors with antigens” could be linked to a list of actual donor names for a particular location. Likewise, the “Patients with antibodies” could be linked to a list of actual patient names for the particular location.

In an example given for illustrative purposes only, a patient with sickle cell anemia will have one or more existing antibodies present in his/her blood. Each time a blood transfusion is made, new antibodies may be introduced into the patient's blood. Since sickle cell anemia requires regular and frequent blood transfusions, it is imperative to be current with the patient's bloodwork.

Embodiments described herein provide an up-to-date blood database for recipients, via the blood database center 110. Therefore, each time a patient receives a blood transfusion, the data for that blood transfusion is updated in the blood database center 110 for the patient. As a result, each time the patient needs another blood transfusion, current data records are immediately available. This is particularly advantageous when the patient is away from his/her home base and regular physicians.

In a fourth embodiment, blood group database tables can be clustered or sub-clustered according to regions of likely need. For example, a clustered blood database for sickle-cell anemia or malaria can be cross-matched with regions in the middle-east and Africa where occurrence of those medical conditions is more prevalent.

The blood donor database 620 for blood donors can have a complete blood make-up on file, which can be cross-matched for a particular medical disease or condition via database server 640. For example, specific blood donors can be cross-matched with one or more clustered database tables as being a positive match for a particular medical disease or condition. Therefore, a first donor might be a positive cross-match with the Hepatitis B blood database and the thrombocytosis blood database. A second donor might be a positive cross-match with the sickle-cell anemia, leukemia, and thrombocytosis blood databases. As a result, in an emergency, a recipient can be quickly matched with available blood of the correct type for his/her condition.

The blood recipient database 630 can be used by frequent blood recipients, such as people needing regular blood transfusions. A future blood recipient can include his/her complete blood workup on file at the blood database center 110. The blood recipient database can be updated to register all information concerning the latest antibodies from each received transfusion. Therefore, in an emergency situation or when the recipient is away from his/her home area, the most recent blood workup information is immediately available, which can also be clustered according to his/her condition(s). This can dramatically speed up finding the proper blood for the recipient because blood testing has already been completed. It also avoids giving the recipient an incorrect blood donation when adequate time is not available to completely process his/her blood testing.

FIG. 6 illustrates the effective and efficient cross-matching of blood donors to blood recipients by database server 640. The registration records of blood recipients can be kept up-to-date with current blood criteria requirements via a blood recipient real-time server. Therefore, the database server 640 can directly match the cross-referenced blood recipient criteria with cross-referenced eligible blood donor criteria, via the blood classification data tables 610. In addition, the database server 640 can cluster and match data from blood donor data tables 620 with the clustered data in blood classification data tables 610, such as by frequency of use, links to a disease or a medical condition, or geographic location association. Likewise, the database server 640 can cluster and match data from blood recipient data tables 630 with the clustered data in blood classification data tables 610, such as by frequency of use, links to a disease or a medical condition, or geographic location association. As a result, a search for a suitable blood donor supply can initially search within a similar or the same cluster as the prospective blood recipient cluster. For example, a blood supply search for a recipient having sickle cell anemia can initially be made within a blood donor data table that is compatible with sickle cell anemia criteria. If a suitable match is not found, a wider search can be made to other blood donor data tables 620.

A web portal is a specially-designed web site that brings information together from diverse information sources to display in a uniform format. A user is presented a uniform web page interface that brings together content from a number of other systems or servers. In some implementations, information related to blood supply requests as well as blood data associated with a current blood supply can be input at the web portal interfaces. FIG. 7A is an illustration of an exemplary blood web portal 120 according to embodiments described herein. However, embodiments described herein are not limited by the particular blood web portal 120 illustrated in FIG. 7A.

Each information source can have a dedicated display region, called a portlet 710. The portlet 710 can depend on the intended user, the intended purpose, implementation framework, and/or code libraries, for example. Blood web portal 120 can include multiple portlets 710ₐ-710ₙ, wherein each portlet 710 can be a dedicated area for each information source. Blood web portal 120 can use a search engine application programming interface (API) to permit users to search via an intranet, as well as extranet content.

Blood web portal 120 can include portlets 710 for blood banks, such as blood bank centers and hospitals; medical service centers, such as hospitals, trauma centers, and emergency response centers; a donor portlet; and a recipient portlet. Blood web portal 120 can also include multiple layers of portlets 710. For example, a blood bank primary portlet can be subdivided into multiple sub-portlets according to specific blood banks or specific regions of blood banks.

The architecture of blood web portal 120 is also illustrated in FIG. 7A. An application server 720 performs most of the functions of the blood web portal 120. In one embodiment, authorized and authenticated users can generate requests to application server 720.

Application server 720 is connected to one or more database servers 730, such as database servers 730ₐ-730ₙ to form a blood availability network. The blood availability network is associated with the blood donor data tables 620 of the blood database center 110. Each database server 730ₐ-730ₙ transmits and receives blood location data to and from a respective portlet 710 of the blood web portal 120. In an embodiment, application server 720 can be part of a clustered server environment. High-capacity portal configurations can include load balancing strategies for the clustered server environment.

A portal server 740 hosts the blood web portal 120 and provides connectivity to the application server 720. In an embodiment, the portal server 740 is a pass-through for a user. Application functionality can be presented in any number of portal pages, via the portlets 710.

FIG. 7B illustrates one embodiment in which each database server 730ₐ-730ₙ serves a particular geographical region in which blood is available. A state, region, or country is divided into portlet quadrants, wherein database server 730ₐ provides blood availability data for the northwest quadrant 710ₐ, database server 730_{b} provides blood availability data for the northeast quadrant 710_{b}, database server 730_{c} provides blood availability data for the southwest quadrant 710_{c}, and database server 730_{d} provides blood availability data for the southeast quadrant 710_{d}.

FIG. 7C illustrates geographical regions in another database server configuration. Each portlet quadrant is served by respective database servers 730ₐ through 730_{d} for each particular type of blood center, such as hospitals 710ₐ, blood banks 710_{b}, trauma centers 710_{c}, and emergency response centers 710_{d}.

In one embodiment, a mobile application for blood web portal 120 is provided. It provides the availability of blood in different locations and the nearest hospital in which the needed blood type is available. In a first example, a mobile application can be customized for a particular user, such as a blood transfusion patient with one or more particular antibodies present in his/her bloodwork. An alarm or notification can be transmitted to the user from the blood web portal 120 when the user is in need of a blood transfusion or when a physician's visit is needed. In addition, the mobile application can locate the nearest cross-matched blood supply for the user, via the application server 720, and transmit the results to the user. Since the user's most recent bloodwork is already present within the blood database center 110, there is no need for manual cross matching prior to locating an acceptable blood supply for the user. Cross-matching a donor's blood with the user is automated and implemented via the blood web portal 120.

FIG. 7D illustrates an exemplary progression for a blood tracking & delivery interface 700 for a mobile application on a mobile device as described by embodiments herein. After surfing to an established web site for the blood tracking and delivery system 100, a first image is displayed, such as image 750 in which a user is requested to login. An option can also be displayed for a new user to register.

After login information is received, multiple choices are displayed in image 760, such as update a profile and find a cross-matched blood supply. In FIG. 7D, the second option to find a cross-matched blood supply is selected.

Multiple locations are searched via a blood tracking and delivery search engine. Image 770 illustrates multiple identified locations in which a cross-matched blood supply is available. A distance from the mobile device is obtained via GPS satellite data in order to find a closest blood supply match to the mobile device. Three locations are illustrated in image 770 in order of distance from the mobile device. In FIG. 7D, the first option of location1 is selected.

Image 780 illustrates an order confirmation in which location1 was selected via the mobile device. FIG. 7D is given for illustrative purposes only. Several other interfaces for a mobile device are contemplated by embodiments described herein.

FIG. 8 is a block diagram of an exemplary blood delivery logistical system 140. FIG. 8 illustrates an identified region in which the requested blood is picked up and delivered to the recipient. The region can be defined by geographical boundaries. In addition, a region can be defined by the vastness of blood supply and delivery networking. For example, a large blood supply and large delivery network can be responsible for a larger region. In a second example, a densely-populated area can be served by several regions in which each region occupies a smaller geographical region. One region can also overlap with one or more other regions.

Using embodiments described herein provides a real-time mapping of where current blood supplies are located and where current blood needs are located. As a result, a more accurate blood network can be developed and implemented.

Blood centers 810 illustrate areas in which blood supplies are located. Blood banks include various blood supplies, which have been obtained from blood donors. The blood supplies have been identified and screened, and are contained for a predetermined time until a recipient has been identified. Hospitals can also include a blood bank for their own use or to be used in cooperation with other hospitals or blood banks.

Service centers 820 are areas in which patients are in need of a particular type and amount of blood. Service centers 820 can include hospitals, Emergency Medical Service (EMS) teams, and within a non-structured field environment such as a natural disaster-hit area, an accident of large magnitude, or an area of civil dispute in which injuries are prevalent.

Unmanned Aerial Vehicle (UAV) network 830 includes an array of UAVs that can pick up and deliver blood supplies from one area to another area. Multiple delivery paths are present between each of the blood centers 810, the service centers 820, and the UAV network 830. In addition, multiple communication paths are present between each of the blood centers 810, the service centers 820, and the UAV network 830.

Blood delivery logistical system 140 is configured to initiate pickup of a cross-matched blood supply at a blood center 810 and delivery of the cross-matched blood supply to a service center 820 in response to a blood supply request. The cross-matched blood supply is identified by the blood database center 110, and the cross-matched blood supply is located by the blood availability network of the blood web portal 120.

Blood delivery logistical system 140 also includes a UAV and a blood delivery container customized for delivery of blood supplies. In one embodiment, the UAV is equipped with one or more refrigeration units to keep the blood supplies cool, especially in high temperature areas. In another embodiment, the blood containers are made with an unbreakable, insulated, and rugged material. This is particularly advantageous when the blood supplies will be air-dropped from the UAV into a casualty site.

FIG. 8 also illustrates a standard delivery network 840 that works in conjunction with the UAV network 830. When blood pickups and deliveries are not possible using standard delivery modes, a request is communicated to the UAV network 830 for pickup or delivery. In addition, when a pickup or delivery was previously not accessible by standard means, but has since become accessible, the pickup and delivery orders can be communicated back to the standard delivery network 840 for completion.

FIG. 8 illustrates segregated bodies of blood centers 810, service centers 820, and the UAV network 830. However, this is just for simplicity of illustration. In an actual blood delivery logistical system 140, each of the blood centers 810, service centers 820, and the UAV network 830 can be inter-twined. FIG. 8 also illustrates just one to two of each blood center 810, service center 820, and UAV network 830. However, hundreds of blood centers 810, service centers 820, and components of UAV network 830 can be present within blood delivery logistical system 140.

In one embodiment, a non-structured field environment service center 820 can be an area of civil unrest or a fighting zone. Therefore, the UAVs may need to have more protective measures taken in order to complete a blood pickup or delivery. For example, attempts may be made to shoot down or incapacitate a UAV that is identified. Therefore, additional armor can be used for the body of the UAV, as well as more rugged electronic devices that are incorporated with the UAV. In addition, modes of communication to and from the UAV can be encrypted to avoid interception. Containers that hold the blood supplies being transported by the UAV can also be more rugged to withstand gunfire and explosives. Using UAVs in an area of civil unrest or in a fighting zone would have a major advantage over sending an individual to make a blood pickup and/or delivery.

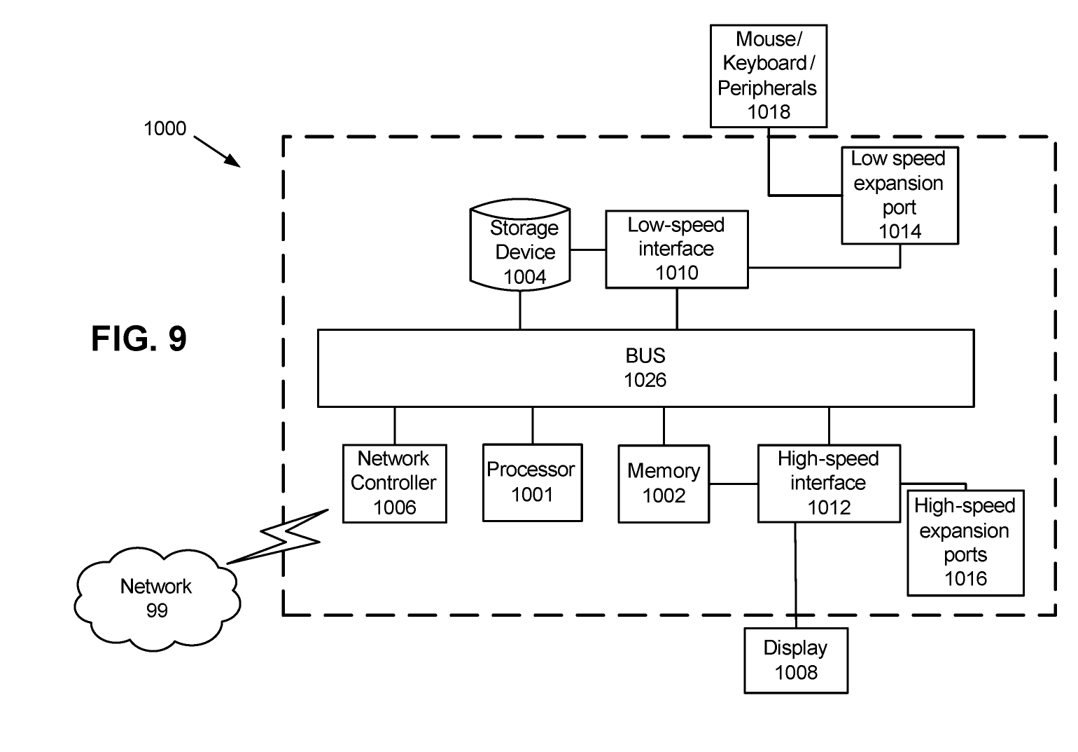

A hardware description of an exemplary computing device 1000 used in accordance with embodiments herein is described with reference to FIG. 9. Computing device 1000 can be used as one or more of the devices illustrated in user transactions to and from the blood database center 110 via the blood organizing engine 110a, the blood web portal 120 via the blood portal engine 120a, the blood hotline 130 via the hotline engine 130a, and/or the blood delivery logistical network 140 via the blood delivery engine 140a illustrated in FIGS. 1A-1B. Computing device 1000 can also be used in transactions between the UAV network 830 and any one of the blood centers 810, the service centers 820, and/or the standard delivery network 840. Computing device 1000 can also be used in transactions between the blood centers 810 and the service centers 820. Computing device 1000 can also be used as the application server 720, the portal server 740, any one of the database servers 730ₐ-730ₙ of the blood web portal 120, any one of the servers 150, the computing device 175 and/or the hotline mobile device terminals 170, which can also be referred to as computing devices. The computing device 1000 can also represent a computing device installed in the UAVs 147 or the device 146 installed within a delivery vehicle that receives commands to distribute blood to one or more predetermined locations.

Computing device 1000 is intended to represent various forms of digital hardware, such as laptops, desktops, workstations, personal digital assistants, servers, blade servers, mainframes, and other appropriate computers. The components shown here, their connections and relationships, and their functions are meant to be examples only and are not meant to be limiting.

The computing device 1000 includes a processor 1001, a memory 1002, a storage device 1004, a high-speed interface 1012 connecting to the memory 1002 and multiple high-speed expansion ports 1016, and a low-speed interface 1010 connecting to a low-speed expansion port 1014 and the storage device 1004. Each of the processor 1001, the memory 1002, the storage device 1004, the high-speed interface 1012, the high-speed expansion ports 1016, and the low-speed interface 1010 are interconnected using various busses, such as communication bus 1026, and may be mounted on a common motherboard or in other manners as appropriate.

The processor 1001 can process instructions for execution within the computing device 1000, including instructions stored in the memory 1002 or on the storage device 1004 to display graphical information for a GUI on an external input/output device, such as a display 1008 coupled to the high-speed interface 1012. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). The memory 1002 stores information within the computing device 1000. In some implementations, the memory 1002 is a volatile memory unit or units. In some implementations, the memory 1002 is a non-volatile memory unit or units. The memory 1002 can also be another form of computer-readable medium, such as a magnetic or optical disk.

The storage device 1004 is capable of providing mass storage for the computing device 1000. In some implementations, the storage device 1004 can be or contain a computer-readable medium, such as a floppy disk device, a hard disk device, an optical disk device, a tape device, a flash memory or other similar solid state memory device, or an array of devices, including devices in a storage area network or other configurations. Instructions can be stored in an information carrier. The instructions, when executed by one or more processing devices (for example, processor 1001), perform one or more methods, such as those described above. The instructions can also be stored by one or more storage devices, such as computer- or machine-readable mediums (for example, the memory 1002, the storage device 1004, or memory on the processor 1001).

The high-speed interface 1012 manages bandwidth-intensive operations for the computing device 1000, while the low-speed interface 1010 manages lower bandwidth-intensive operations. Such allocation of functions is an example only. In some implementations, the high-speed interface 1012 is coupled to the memory 1002, the display 1008 (e.g., through a graphics processor or accelerator), and to the high-speed expansion ports 1016, which may accept various expansion cards (not shown). In the implementation, the low-speed interface 1010 is coupled to the storage device 1004 and the low-speed expansion port 1014. The low-speed expansion port 1014, which can include various communication ports (e.g., USB, Bluetooth, Ethernet, wireless Ethernet) can be coupled to one or more input/output devices 1018, such as a keyboard, a pointing device, a scanner, or a networking device such as a switch or router, e.g., through a network adapter.

The computing device 1000 also includes a network controller 1006, such as an Intel Ethernet PRO network interface card from Intel Corporation of America, for interfacing with a network 99. As can be appreciated, the network 99 can be a public network, such as the Internet, or a private network such as an LAN or WAN network, or any combination thereof and can also include PSTN or ISDN sub-networks. The network 99 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

Although the computing device 1000 of FIG. 9 is described as having a storage medium device 1004, the claimed advancements are not limited by the form of the computer-readable media on which the instructions of the described processes are stored. For example, the instructions can be stored on CDs, DVDs, in FLASH memory, RAM, ROM, PROM, EPROM, EEPROM, hard disk, or any other information processing device with which the computing device communicates.

In other alternate embodiments, processing features according to the present disclosure may be implemented and commercialized as hardware, a software solution, or a combination thereof. Moreover, instructions corresponding to processes described herein could be stored in a portable drive, such as a USB Flash drive that hosts a secure process.

Computer programs (also known as programs, software, software applications, or code) associated with the processes described herein include machine instructions for a programmable processor, and can be implemented in a high-level procedural and/or object-oriented programming language, and/or in assembly/machine language. As used herein, the terms machine-readable medium and computer-readable medium refer to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described herein can be implemented on a computer having a display device 1008 (e.g., a CRT (cathode ray tube) or LCD (liquid crystal display) monitor) for displaying information to the user and a keyboard and a pointing device 1018 (e.g., a mouse or a trackball) by which the user can provide input to the computer. Other kinds of devices can be used to provide for interaction with a user as well. For example, feedback provided to the user can be any form of sensory feedback (e.g., visual feedback, auditory feedback, or tactile feedback), and input from the user can be received in any form, including acoustic, speech, or tactile input.

The systems and techniques described herein can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

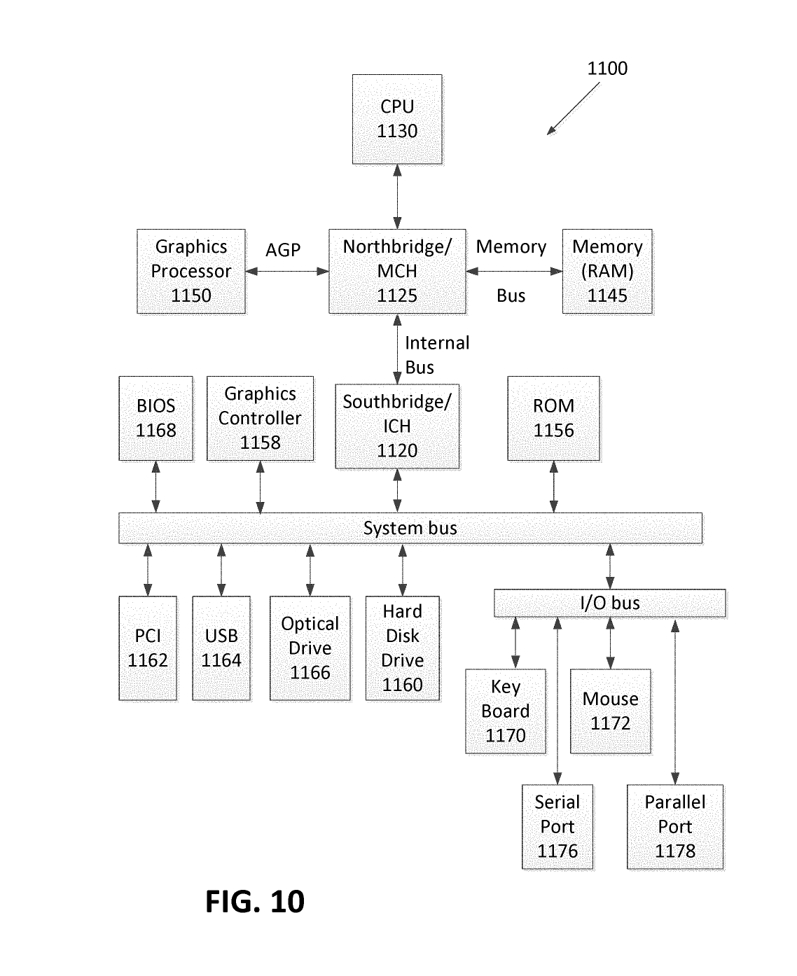

FIG. 10 shows a schematic diagram of an exemplary data processing system, according to aspects of the disclosure described herein for performing menu navigation, as described above. The data processing system is an example of a computer in which code or instructions implementing the processes of the illustrative embodiments can be located.

In FIG. 10, data processing system 1100 employs an application architecture including a north bridge and memory controller hub (NB/MCH) 1125 and a south bridge and input/output (I/O) controller hub (SB/ICH) 1120. The central processing unit (CPU) 1130 is connected to NB/MCH 1125. The NB/MCH 1125 also connects to the memory 1145 via a memory bus, and connects to the graphics processor 1150 via an accelerated graphics port (AGP). The NB/MCH 1125 also connects to the SB/ICH 1120 via an internal bus (e.g., a unified media interface or a direct media interface). The CPU 1130 can contain one or more processors and even can be implemented using one or more heterogeneous processor systems.

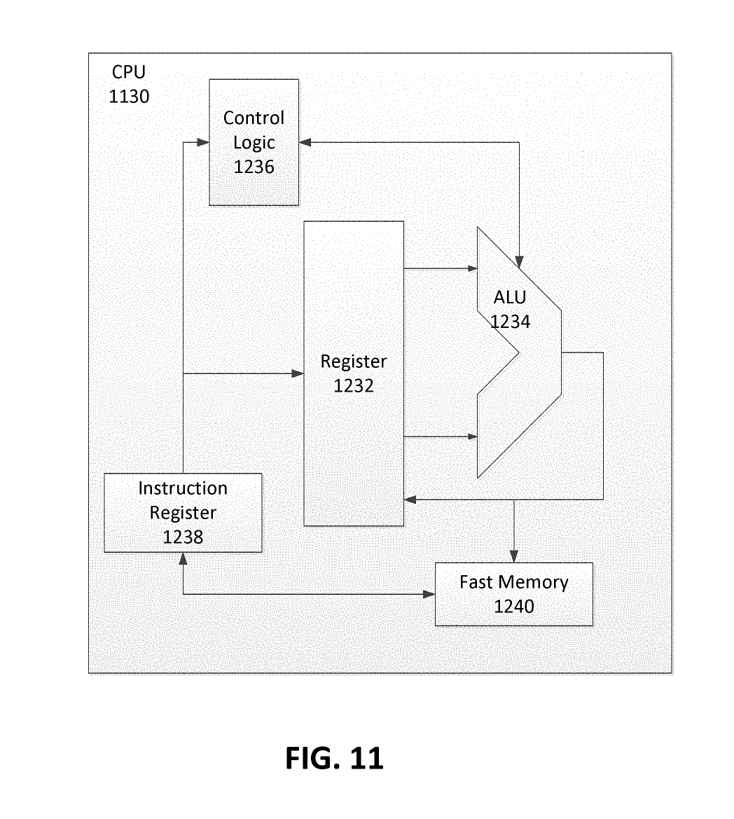

For example, FIG. 11 illustrates one implementation of CPU 1130. In one implementation, an instruction register 1238 retrieves instructions from a fast memory 1240. At least part of these instructions are fetched from an instruction register 1238 by a control logic 1236 and interpreted according to the instruction set architecture of the CPU 1130. Part of the instructions can also be directed to a register 1232. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using an arithmetic logic unit (ALU) 1234 that loads values from the register 1232 and performs logical and mathematical operations on the loaded values according to the instructions. The results from these operations can be fed back into the register 1232 and/or stored in a fast memory 1240. According to aspects of the disclosure, the instruction set architecture of the CPU 1130 can use a reduced instruction set computer (RISC), a complex instruction set computer (CISC), a vector processor architecture, or a very long instruction word (VLIW) architecture. Furthermore, the CPU 1130 can be based on the Von Neuman model or the Harvard model. The CPU 1130 can be a digital signal processor, an FPGA, an ASIC, a PLA, a PLD, or a CPLD. Further, the CPU 1130 can be an x86 processor by Intel or by AMD; an ARM processor; a Power architecture processor by, e.g., IBM; a SPARC architecture processor by Sun Microsystems or by Oracle; or other known CPU architectures.

Referring again to FIG. 10, the data processing system 1100 can include the SB/ICH 1120 being coupled through a system bus to an I/O Bus, a read only memory (ROM) 1156, universal serial bus (USB) port 1164, a flash binary input/output system (BIOS) 1168, and a graphics controller 1158. PCI/PCIe devices can also be coupled to SB/ICH 1120 through a PCI bus 1162.

The PCI devices can include, for example, Ethernet adapters, add-in cards, and PC cards for notebook computers. The Hard disk drive 1160 and CD-ROM 1166 can use, for example, an integrated drive electronics (IDE) or serial advanced technology attachment (SATA) interface. In one implementation the I/O bus can include a super I/O (SIO) device.

Further, the hard disk drive (HDD) 1160 and optical drive 1166 can also be coupled to the SB/ICH 1120 through a system bus. In one implementation, a keyboard 1170, a mouse 1172, a parallel port 1178, and a serial port 1176 can be connected to the system bus through the I/O bus. Other peripherals and devices can be connected to the SB/ICH 1120 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

The functions and features described herein can also be executed by various distributed components of a system. For example, one or more processors can execute these system functions, wherein the processors are distributed across multiple components communicating in a network. The distributed components can include one or more client and server machines, which can share processing, such as a cloud computing system, in addition to various human interface and communication devices (e.g., display monitors, smart phones, tablets, personal digital assistants (PDAs)). The network can be a private network, such as a LAN or WAN, or can be a public network, such as the Internet. Input to the system can be received via direct user input and received remotely either in real-time or as a batch process. Additionally, some implementations can be performed on modules or hardware not identical to those described. Accordingly, other implementations are within the scope that can be claimed.

The functions and features described herein may also be executed by various distributed components of a system. For example, one or more processors may execute these system functions, wherein the processors are distributed across multiple components communicating in a network. For example, distributed performance of the processing functions can be realized using grid computing or cloud computing. Many modalities of remote and distributed computing can be referred to under the umbrella of cloud computing, including: software as a service, platform as a service, data as a service, and infrastructure as a service. Cloud computing generally refers to processing performed at centralized locations and accessible to multiple users who interact with the centralized processing locations through individual terminals.

Embodiments described herein can be implemented in conjunction with one or more of the devices described above with reference to FIGS. 9-11. Embodiments are a combination of hardware and software, and processing circuitry by which the software is implemented.

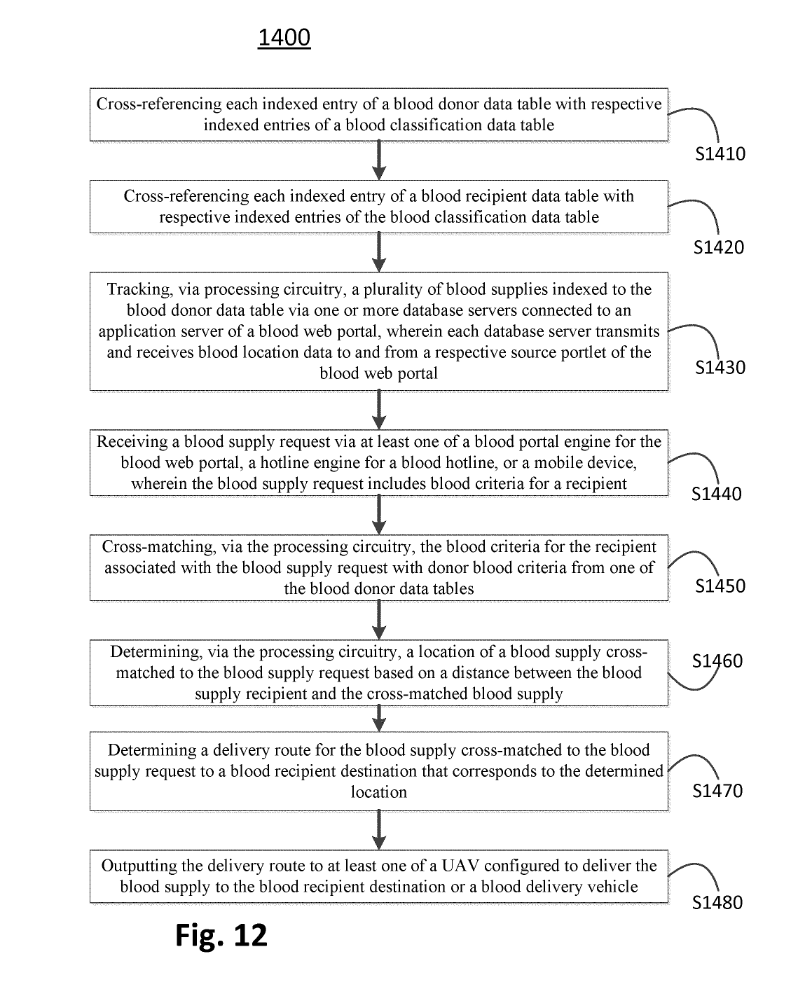

FIG. 12 illustrates an exemplary algorithmic flowchart for a method 1400 of tracking and delivering blood according to an aspect of the present disclosure. Method 1400 includes programmable computer-executable instructions, that when used in combination with the above-described hardware devices, carry out the steps of method 1400. The hardware description above, exemplified by any one of the structural examples illustrated in FIGS. 9-11 constitutes or includes specialized corresponding structure that is programmed or configured to perform the algorithm illustrated in FIG. 12. For example, the algorithm illustrated in FIG. 12 can be completely performed by the single device illustrated in FIG. 9 or the chipset illustrated in FIG. 10.

FIG. 12 is a flowchart for an exemplary method 1400 of tracking and delivering blood. Method 1400 can be implemented using one or more of the computing systems 1000 or 1100 described herein.

In step S1410, each indexed entry of a blood donor data table is cross-referenced with respective indexed entries of a blood classification data table, via a blood organizing engine of a blood database center, such as blood database center 110. For example, a donor with AB+ blood would be indexed to the ABO System 001 Antigen Number 003, and Rh System 004 Antigen Number 001. Any other antigens present in the donor's blood would also be indexed to the respective system(s) and antigen number(s) of the blood classification data table.

In step S1420, each indexed entry of a blood recipient data table is cross-referenced with respective indexed entries of the blood classification data table. The blood systems and antigens present in the recipient's blood are cross-referenced to the blood classification data table, similar to a donor's cross-referencing. In addition, any requirements for the absence of particular antigens are also cross-referenced to the blood classification data table.

In step S1430, a plurality of blood supplies indexed to the blood donor data table is tracked, via one or more database servers connected to an application server of a blood web portal, such as blood web portal 120. Each database server transmits and receives blood location data to and from a respective source portlet of the blood web portal. For example, a source portlet representing a hospital in Washington, D.C. is holding a particular blood supply linked to the blood donor data table in blood database center 110. Therefore, the specific blood data is linked to the location of the actual blood supply.

In step S1440, a blood supply request is received via at least one of a blood portal engine for the blood web portal, a hotline engine for a blood hotline, or a mobile device. The blood supply request includes blood criteria for a recipient. The blood supply request can be initiated by medical personnel at a service center to the blood web portal 120 to make a specific blood supply request. The request can also be initiated via a blood hotline, such as blood hotline 130. In another example, a request can be made directly from a user, via a mobile application of the blood web portal 120.

In step S1450, the blood criteria for the recipient associated with the blood supply request is cross matched with donor blood criteria from one of the blood donor data tables, via the blood organizing engine. In an example, the recipient of the blood request may be a registered user with the blood recipient data table, and is in present need for a blood transfusion. Since the recipient's current blood workup is already known, an immediate search for a matching donor can be initiated.

In step S1460, a location of a blood supply cross-matched to the blood supply request based on a distance between the blood supply recipient and the cross-matched blood supply, via the blood portal engine. A location of the recipient is known through the original request, or a GPS location can be determined for a mobile application request.

In step S1470, a delivery route is determined for the blood supply cross-matched to the blood supply request to a blood recipient destination that corresponds to the determined location, via a blood delivery engine. When the communications network has determined that an expedited blood delivery beyond current standard delivery means is necessary, a UAV delivery path is established for pickup of the blood supply at the blood center and delivery of the blood supply to the service center. In an example, the service center can be an unstructured field area, such as a disaster-hit area or an area of civil or military unrest.

In step S1480, the delivery route is outputted to at least one of a UAV configured to deliver the blood supply to a blood recipient destination or a blood delivery vehicle. In one embodiment, the delivery route control signal is outputted to a UAV to control the route taken to the delivery location. In a second embodiment, the delivery route control signal outputs location information to the UAV or other transportation vehicle, and the UAV or other transportation vehicle determines a specific route to be taken.

Embodiments described herein provide several technical advantages. Blood database center 110 includes blood donor data tables 620 that are cross-referenced to applicable blood classification data tables 610. For example, blood criteria of each donor in the blood donor data tables 620 are cross-referenced to respective blood system and antigen data in the blood classification data tables 610. Likewise, blood recipient data tables 630 are cross-referenced to applicable blood classification data tables 610. In addition, each type of blood data table can be clustered according to a frequency of use, disease or medical condition, or geographic location association, for example. This provides a faster and more efficient database of cross-matching between donors and recipients. In addition, the database cross-matching is more accurate and complete using embodiments for blood database center 110 described herein.

Blood web portal 120 provides technical advantages also. Each information source is represented in blood web portal 120 by its own portlet 710, wherein all portlets 710 are displayed in a uniform format within the same online location. Database servers provide a faster and more thorough search and identification for the location of available blood supplies.

Blood delivery logistical system 140 provides a comprehensive connection of blood supply transportation. A UAV network 830 provides fast and direct pickup and delivery of identified blood supplies from a blood center 810 to a service center 820, regardless of current road or traffic conditions. A service center 820 can also include a non-structured field environment, such as a disaster-hit area or an area of civil or military unrest. The UAV network 830 is also connected to a standard delivery network 840 when standard transportation is a viable option.

Blood tracking and delivery device 100 provides several technical advantages of providing blood supply needs over conventional blood supply networks. It can be applied to almost any environment in which ground transportation or helicopter delivery is not an option, which ultimately save more lives.

Embodiments described herein include the following aspects.

(1) A blood tracking and delivery system, including: at least one unmanned aerial vehicle (UAV) configured to carry a blood supply to a blood recipient destination; and a blood tracking and delivery system server having processing circuitry configured to receive blood data at a blood database center, wherein a) the blood data is sorted and indexed into a plurality of database tables including at least one of a blood donor database table, at least one blood classification data table, or a blood recipient data table, b) each entry of the blood donor data table is cross-referenced to respective indexed entries of the at least one blood classification data table, and c) each indexed entry of the blood recipient data table is cross-referenced to respective indexed entries of the at least one blood classification data table, receive a blood supply request from at least one computing device, the blood supply request including blood criteria for a recipient, cross-match the blood criteria for the recipient associated with the blood supply request with donor blood criteria from the blood donor data table, determine a location of a blood supply cross-matched to the blood supply request based on a distance between the blood supply recipient and the cross-matched blood supply, and determine a delivery route for the blood supply cross-matched to the blood supply request to a blood recipient destination that corresponds to the determined location, wherein the UAV is configured to deliver the blood supply to the blood recipient destination.

(2) The blood tracking and delivery system of (1), wherein the at least one blood classification data table includes one or more blood system groups and associated antigen groups.

(3) The blood tracking and delivery system of (1) or (2), wherein the processing circuitry is further configured to index at least one blood classification data table based on at least one of frequency of use, links to a disease or a medical condition, or geographic location association.

(4) The blood tracking and delivery system of any one of (1) to (3), wherein the processing circuitry is further configured to index the blood donor data table and the blood recipient data table based on at least one of frequency of use, links to a disease or a medical condition, or geographic location association when cross-referenced with a similarly indexed blood classification data table.

(5) The blood tracking and delivery system of any one of (1) to (4), wherein the processing circuitry is further configured to host a blood web portal including a plurality of portlets, each portlet representing an information source entity.

(6) The blood tracking and delivery system of any one of (1) to (5), wherein the processing circuitry is configured to receive the blood supply request from a blood web portal mobile application of the computing device.

(7) The blood tracking and delivery system of any one of (1) to (6), wherein the blood tracking and delivery system server is communicatively connected to an unmanned aerial vehicle network for logistical transport of the blood supply cross-matched to the blood supply request for delivery to the blood recipient destination.

(8) The blood tracking and delivery system of any one of (1) to (7), wherein the processing circuitry is further configured to output at least one blood web portal interface to the at least one computing device, and receive at least one of the blood supply request or the blood data associated with a current blood supply.

(9) The blood tracking and delivery system of any one of (1) to (8), wherein the at least one blood web portal interface includes an information source for one of a hospital and a blood bank.

(10) The blood tracking and delivery system of any one of (1) to (9), wherein the at least one blood web portal interface includes an information source for one of an emergency medical service team and a non-structured field environment.

(11) A method of tracking and delivering blood, the method including: cross-referencing each indexed entry of a blood donor data table with respective indexed entries of a blood classification data table; cross-referencing each indexed entry of a blood recipient data table with respective indexed entries of the blood classification data table; tracking, via processing circuitry, a plurality of blood supplies indexed to the blood donor data table via one or more database servers connected to an application server of a blood web portal, wherein each database server transmits and receives blood location data to and from a respective source portlet of the blood web portal; receiving a blood supply request from at least one computing device, the blood supply request including blood criteria for a recipient; cross-matching, via the processing circuitry, the blood criteria for the recipient associated with the blood supply request with donor blood criteria from one of the blood donor data tables; determining, via the processing circuitry, a location of a blood supply cross-matched to the blood supply request based on a distance between the blood supply recipient and the cross-matched blood supply; and determining a delivery route for the blood supply cross-matched to the blood supply request to a blood recipient destination that corresponds to the determined location, wherein an unmanned aerial vehicle (UAV) is configured to deliver the blood supply to the blood recipient destination.

(12) The method of (11), wherein the blood classification data table includes multiple blood system groups and associated antigen groups.

(13) The method of (11) or (12), further including: indexing the blood classification data table according to one or more of frequency of use, links to a disease or a medical condition, and geographic location association.

(14) The method of any one of (11) to (13), further including: indexing the blood donor data table and the blood recipient data table according to one or more of frequency of use, links to a disease or a medical condition, and geographic location association when cross-referenced with a similarly indexed blood classification data table.

(15) The method of any one of (11) to (14), wherein the respective source portlet of the blood web portal represents an information source entity.

(16) The method of any one of (11) to (15), further including: hosting the blood web portal on a portal server and providing connectivity to the application server via the portal server.

(17) The method of any one of (11) to (16), further including: receiving the blood supply request from the computing device via a blood hotline having direct access to the blood web portal.

(18) The method of any one of (11) to (17), further including: providing non-UAV logistical transport of the cross-matched blood supply via a standard delivery network communicatively connected to the blood delivery logistical network for UAV transportation.

(19) A non-transitory computer-readable media having computer-executable instructions embodied thereon, that when executed by a first computing device, performs a method including: receiving blood data at a blood database center, wherein a) the blood data is sorted and indexed into a plurality of database tables including at least one of a blood donor database table, at least one blood classification data table, or a blood recipient data table, b) each entry of the blood donor data table is cross-referenced to respective indexed entries of the at least one blood classification data table, and c) each indexed entry of the blood recipient data table is cross-referenced to respective indexed entries of the at least one blood classification data table; receiving a blood supply request from at least one second computing device, the blood supply request including blood criteria for a recipient; cross-matching the blood criteria for the recipient associated with the blood supply request with donor blood criteria from one of the blood donor data tables; determining a location of a blood supply cross-matched to the blood supply request based on a distance between the blood supply recipient and the cross-matched blood supply; and determining a delivery route for the blood supply cross-matched to the blood supply request to a blood recipient destination that corresponds to the determined location, wherein an unmanned aerial vehicle (UAV) is configured to deliver the blood supply to the blood recipient destination.

(20) The non-transitory computer-readable media of (19), wherein the at least one blood classification data table includes multiple blood system groups and associated antigen groups.

A number of implementations have been described. Nevertheless, it will be understood that various modifications may be made without departing from the spirit and scope of this disclosure. For example, preferable results may be achieved if the steps of the disclosed techniques were performed in a different sequence, if components in the disclosed systems were combined in a different manner, or if the components were replaced or supplemented by other components. The functions, processes, and algorithms described herein may be performed in hardware or software executed by hardware, including computer processors and/or programmable circuits configured to execute program code and/or computer instructions to execute the functions, processes, and algorithms described herein. Additionally, an implementation may be performed on modules or hardware not identical to those described. Accordingly, other implementations are within the scope that may be claimed.

The foregoing discussion describes merely exemplary embodiments of the present disclosure. As will be understood by those skilled in the art, the present disclosure may be embodied in other specific forms without departing from the spirit or essential characteristics thereof. Accordingly, the disclosure is intended to be illustrative, but not limiting of the scope of the disclosure, as well as the claims. The disclosure, including any readily discernible variants of the teachings herein, defines in part, the scope of the foregoing claim terminology such that no inventive subject matter is dedicated to the public.

1. A blood tracking and delivery system, comprising:
at least one unmanned aerial vehicle (UAV) configured to carry a blood supply to a blood recipient destination; and
a blood tracking and delivery system server having processing circuitry configured to
receive blood data at a blood database center, wherein a) the blood data is sorted and indexed into a plurality of database tables including at least one of a blood donor database table, at least one blood classification data table, or a blood recipient data table, b) each entry of the blood donor data table is cross-referenced to respective indexed entries of the at least one blood classification data table, and c) each indexed entry of the blood recipient data table is cross-referenced to respective indexed entries of the at least one blood classification data table,
receive a blood supply request from at least one computing device, the blood supply request including blood criteria for a recipient,
cross-match the blood criteria for the recipient associated with the blood supply request with donor blood criteria from the blood donor data table,
determine a location of a blood supply cross-matched to the blood supply request based on a distance between the blood supply recipient and the cross-matched blood supply, and
determine a delivery route for the blood supply cross-matched to the blood supply request to a blood recipient destination that corresponds to the determined location, wherein
the UAV is configured to deliver the blood supply to the blood recipient destination based on the delivery route.

2. The blood tracking and delivery system of claim 1, wherein the at least one blood classification data table includes one or more blood system groups and associated antigen groups.

3. The blood tracking and delivery system of claim 2, wherein the processing circuitry is further configured to index at least one blood classification data table based on at least one of frequency of use, links to a disease or a medical condition, or geographic location association.

4. The blood tracking and delivery system of claim 3, wherein the processing circuitry is further configured to index the blood donor data table and the blood recipient data table based on at least one of frequency of use, links to a disease or a medical condition, or geographic location association when cross-referenced with a similarly indexed blood classification data table.

5. The blood tracking and delivery system of claim 1, wherein the processing circuitry is further configured to host a blood web portal including a plurality of portlets, each portlet representing an information source entity.

6. The blood tracking and delivery system of claim 1, wherein the processing circuitry is configured to receive the blood supply request from a blood web portal mobile application of the computing device.

7. The blood tracking and delivery system of claim 1, wherein the blood tracking and delivery system server is communicatively connected to an unmanned aerial vehicle network for logistical transport of the blood supply cross-matched to the blood supply request for delivery to the blood recipient destination.

8. The blood tracking and delivery system of claim 1, wherein the processing circuitry is further configured to
output at least one blood web portal interface to the at least one computing device, and
receive at least one of the blood supply request or the blood data associated with a current blood supply.

9. The blood tracking and delivery system of claim 8, wherein the at least one blood web portal interface includes an information source for one of a hospital and a blood bank.

10. The blood tracking and delivery system of claim 8, wherein the at least one blood web portal interface includes an information source for one of an emergency medical service team and a non-structured field environment.

11. A method of tracking and delivering blood, the method comprising:
cross-referencing each indexed entry of a blood donor data table with respective indexed entries of a blood classification data table;
cross-referencing each indexed entry of a blood recipient data table with respective indexed entries of the blood classification data table;
tracking, via processing circuitry, a plurality of blood supplies indexed to the blood donor data table via one or more database servers connected to an application server of a blood web portal, wherein each database server transmits and receives blood location data to and from a respective source portlet of the blood web portal;
receiving a blood supply request from at least one computing device, the blood supply request including blood criteria for a recipient;
cross-matching, via the processing circuitry, the blood criteria for the recipient associated with the blood supply request with donor blood criteria from one of the blood donor data tables;
determining, via the processing circuitry, a location of a blood supply cross-matched to the blood supply request based on a distance between the blood supply recipient and the cross-matched blood supply; and
determining a delivery route for the blood supply cross-matched to the blood supply request to a blood recipient destination that corresponds to the determined location, wherein
an unmanned aerial vehicle (UAV) is configured to deliver the blood supply to the blood recipient destination based on the delivery route.

12. The method of claim 11, wherein the blood classification data table includes multiple blood system groups and associated antigen groups.

13. The method of claim 12, further comprising:
indexing the blood classification data table according to one or more of frequency of use, links to a disease or a medical condition, and geographic location association.

14. The method of claim 13, further comprising:
indexing the blood donor data table and the blood recipient data table according to one or more of frequency of use, links to a disease or a medical condition, and geographic location association when cross-referenced with a similarly indexed blood classification data table.

15. The method of claim 11, wherein the respective source portlet of the blood web portal represents an information source entity.

16. The method of claim 15, further comprising:
hosting the blood web portal on a portal server and providing connectivity to the application server via the portal server.

17. The method of claim 11, further comprising:
receiving the blood supply request from the computing device via a blood hotline having direct access to the blood web portal.

18. The method of claim 11, further comprising:
providing non-UAV logistical transport of the cross-matched blood supply via a standard delivery network communicatively connected to the blood delivery logistical network for UAV transportation.

19. A non-transitory computer-readable media having computer-executable instructions embodied thereon, that when executed by a first computing device, performs a method comprising:
receiving blood data at a blood database center, wherein a) the blood data is sorted and indexed into a plurality of database tables including at least one of a blood donor database table, at least one blood classification data table, or a blood recipient data table, b) each entry of the blood donor data table is cross-referenced to respective indexed entries of the at least one blood classification data table, and c) each indexed entry of the blood recipient data table is cross-referenced to respective indexed entries of the at least one blood classification data table;
receiving a blood supply request from at least one second computing device, the blood supply request including blood criteria for a recipient;
cross-matching the blood criteria for the recipient associated with the blood supply request with donor blood criteria from one of the blood donor data tables;
determining a location of a blood supply cross-matched to the blood supply request based on a distance between the blood supply recipient and the cross-matched blood supply; and
determining a delivery route for the blood supply cross-matched to the blood supply request to a blood recipient destination that corresponds to the determined location, wherein
an unmanned aerial vehicle (UAV) is configured to deliver the blood supply to the blood recipient destination based on the delivery route.

20. The non-transitory computer-readable media of claim 19, wherein the at least one blood classification data table includes multiple blood system groups and associated antigen groups.
